(19) United States
(12) Patent Application Publication
(10) Pub. No.: US 2004/0122391 A1
(43) Pub. Date: Jun. 24, 2004

(54) Apparatus and method for magnetically controlling a moving web of material

(75) Inventor: Kent A. Franklin, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/328,534

(22) Filed: Dec. 23, 2002

(51) Int. Cl. A61F 13/15; A61F 13/20

(52) U.S. Cl. 604/367

(57) ABSTRACT

In a method and apparatus for controlling at least one of the position, the orientation and the configuration of a material web during movement of the web in a predetermined direction, a magnetically responsive material is applied to the material web and the web is exposed to at least one magnetic field during movement of the web in the predetermined direction. The at least one magnetic field attracts or repels the magnetically responsive material applied to the material web to thereby control at least one of the position, the orientation and the configuration of the web during movement of the web in the predetermined direction. In one embodiment, the magnetic field is generated by a magnetic field generator operable to generate a magnetic field in magnetically responsive communication with the material web upon movement of the web in the predetermined direction.

### BACKGROUND OF THE INVENTION

The present invention relates generally to apparatus and methods for controlling the position, orientation and/or configuration of a web of material, such as a continuous web or a discrete web of material, and more particularly to apparatus and methods for magnetically controlling a moving web of material.

Material webs, whether continuous or discrete, are often used in the manufacture of garments, and more particularly disposable absorbent garments. Such garments have numerous applications including diapers, training pants, feminine care products, medical garments, adult incontinence products and the like. The material webs used to make these garments may, e.g., be a film, a sheet, a non-woven, a laminate or other flexible web. A typical disposable absorbent garment is formed as a composite structure including at least an absorbent structure disposed between material webs such as a liquid permeable inner web, also commonly referred to as a bodyside liner, and a liquid impermeable outer web, also commonly referred to as an outer cover.

These components can be combined with other webs and components such as elastic materials and containment structures to form a product which is specifically suited to its intended purposes. A number of such garments also include fastening components which are intended to be secured together (e.g., pre-fastened) during manufacture of the garment so that the product is packaged in its fully assembled form. For example, one pre-fastened garment is a pair of children's training pants, which has a central absorbent chassis including the inner web, the outer web, an absorbent structure therebetween, and front and rear side panels (the side panels also being material webs) extending laterally out from each side of the absorbent chassis. Each of the front and rear side panels has a fastening component thereon, such as a hook or a loop type fastener.

To manufacture absorbent garments such as the above-described pair of training pants, particularly in a high-speed manufacturing process, the central absorbent chassis is initially formed by cutting, positioning, orienting and configuring of the various material webs and other components used to make the garment so as to assemble and secure the material webs and components in a desired arrangement. For example, apparatus for manufacturing garments such as training pants typically includes various rolls, conveyors and other devices used to position, orient and configure material webs as the webs are transported in the direction of assembly of the training pants.

Various apparatus are available to manipulate or otherwise control a continues or discrete web of material, e.g., to position, orient and/or configure such a material web, as the web is moved by a manufacturing apparatus in the direction of assembly. For example, such apparatus typically control the position, orientation and configuration of moving webs pneumatically, such as by using vacuum or pressurized air acting on the web. Mechanical tensioning devices, roller/nip systems and belts are also commonly used to the control material webs during movement of such web components in the direction of assembly of an absorbent garment.

### SUMMARY OF THE INVENTION

In general, one embodiment of a method of the present invention for controlling at least one of the position, the orientation and the configuration of a material web during movement of the web in a predetermined direction comprises applying a magnetically responsive material to the material web and exposing the material web to at least one magnetic field during movement of the web in the predetermined direction. The at least one magnetic field attracts or repels the magnetically responsive material applied to the material web to thereby control at least one of the position, the orientation and the configuration of the web during movement of the web in the predetermined direction.

One embodiment of a disposable article of the present invention generally comprises at least one material web. The material web has a magnetically responsive material applied thereto.

Apparatus according to one embodiment of the present invention for magnetically controlling at least one of the position, the orientation and the configuration of a material web having a magnetically responsive material applied thereto upon movement of the web in a predetermined direction generally comprises at least one magnetic field generator operable to generate a magnetic field in magnetically responsive communication with the material web upon movement of the web in the predetermined direction. The magnetic field is capable of one of attracting and repelling the magnetically responsive material applied to the material web.

### BRIEF DESCRIPTION OF THE DRAWINGS

FIG. 1 is a schematic of apparatus for making garments such as training pants wherein the apparatus includes apparatus for magnetically controlling a moving web used in making such garments;

FIG. 2 is a side view of a knife roll, anvil roll and transfer roll of the apparatus of FIG. 1 with an end wall of the anvil roll omitted to show internal construction of the anvil roll;

FIG. 2a is a front elevation of the anvil roll of FIG. 2 with a material web moving over the anvil roll;

FIG. 2b is a front elevation of an alternative embodiment of the anvil roll of FIG. 2;

FIG. 2c is a front elevation of another alternative embodiment of the anvil roll of FIG. 2;

FIG. 3 is a side view similar to FIG. 2 showing another embodiment of the anvil roll;

FIG. 4 is a top plan view of a roll over which a moving web passes upon movement of the web in a predetermined direction, and a pair of magnetic field generators positioned upstream of the roll;

FIG. 4a is a rear elevation of the roll of FIG. 4 illustrating one embodiment of the magnetic field generators;

FIG. 4b is a rear elevation of the roll of FIG. 4 illustrating another embodiment of the magnetic field generators; and

FIG. 5 is another embodiment of apparatus for making garments such as training pants wherein the apparatus includes apparatus for magnetically controlling a moving web used in making such garments.

Corresponding parts are designated by corresponding reference characters and numerals throughout the several views of the drawings.

Definitions

Within the context of this specification, each term or phrase below will include the following meaning or meanings.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Comprising” is inclusive or open-ended and does not exclude additional, unrecited elements or method steps.

“Connected” refers to the joining, adhering, bonding, attaching, or the like, of two elements. Two elements will be considered to be connected together when they are connected directly to one another or indirectly to one another, such as when each is directly connected to intermediate elements.

“Disposable” refers to articles which are designed to be discarded after a limited use rather than being laundered or otherwise restored for reuse.

“Disposed, disposed on,” and variations thereof are intended to mean that one element can be integral with another element, or that one element can be a separate structure bonded to or placed with or placed near another element.

“Elastic, elasticized and elasticity” mean that property of a material or composite by virtue of which it tends to recover its original size and shape after removal of a force causing a deformation.

“Fabrics” is used to refer to all of the woven, knitted and nonwoven fibrous webs.

“Film” refers to a thermoplastic film made using a film extrusion and/or foaming process, such as a cast film or blown film extrusion process. The term includes apertured films, slit films, and other porous films which constitute liquid transfer films, as well as films which do not transfer liquid.

“Flexible” refers to materials which are compliant and which will readily conform to the general shape and contours of the wearer's body.

“Force” includes a physical influence exerted by one body on another which produces acceleration of bodies that are free to move and deformation of bodies that are not free to move.

“Integral” is used to refer to various portions of a single unitary element rather than separate structures bonded to or placed with or placed near one another.

“Inward” and “outward” refer to positions relative to the center of an article, and particularly transversely and/or longitudinally closer to or away from the longitudinal and transverse center of the article.

“Layer,” when used in the singular, can have the dual meaning of a single element or a plurality of elements.

“Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, may spread or be transported parallel to the plane of the liquid impermeable layer or laminate, but this is not considered to be within the meaning of “liquid impermeable when used herein.

“Longitudinal” and “transverse” have their customary meanings. The longitudinal axis lies in the plane of the article, or a material web or component thereof, and is generally parallel to a vertical plane that bisects a standing wearer into left and right body halves when the article is worn. The transverse axis lies in the plane of the article, or a material web or component thereof, generally perpendicular to the longitudinal axis.

“Member,” when used in the singular, can have the dual meaning of a single element or a plurality of elements.

“Nonwoven” and “nonwoven web” refer to materials and webs of material which are formed without the aid of a textile weaving or knitting process. The web has a structure of individual fibers or threads which are interlaid, but not in an identifiable manner as in a knitted fabric. Non-woven webs have been formed from many processes such as, for example, meltblowing processes, spunbonding processes, and bonded-carded processes.

“Stretch bonded” refers to an elastic member being bonded to another member while the elastic member is extended at least about 25 percent of its relaxed length. Desirably, the term stretch bonded refers to the situation wherein the elastic member is extended at least about 100 percent, and more desirably at least about 300 percent, of its relaxed length when it is bonded to the other member.

“Tension” includes a uniaxial force tending to cause the extension of a body or the balancing force within that body resisting the extension.

“Thermoplastic” describes a material that softens when exposed to heat and which substantially returns to a nonsoftened condition when cooled to room temperature.

These terms may be defined with additional language in the remaining portions of the specification.

### DETAILED DESCRIPTION

The present invention, as described in further detail below, is generally directed to apparatus and methods for controlling or otherwise manipulating a material web, such as the position, orientation and configuration of such a web, as the web moves in a predetermined direction. The material web may be constructed of substantially any flexible fabric, such as a woven web, a knitted web or a non-woven web, or the web may be a film, sheet or any other flexible material. The web may be stretchable, such as either elastic or extensible, or it may be non-stretchable. It is also contemplated that the material web may be a laminate, composite or other combination of any of the above materials.

In accordance with the present invention, the web of material has a magnetically responsive material applied thereto. Upon movement of the web in a predetermined direction, the web is exposed to one or more magnetic fields generated by one or more suitable magnetic field generators to control or otherwise manipulate the web, e.g., to position, orient and/or configure the web as desired. As used herein, the term “position” is used to refer to the spatial position (e.g., height, longitudinal and/or lateral position, etc.) of the material web, for example, relative to another web, or to an article being assembled or a component of such an article, or to apparatus used to transport the web in a predetermined direction. The term “orientation” is used to refer to the orientation (e.g., rotation, skew, angle) of the web, for example, relative to another web, or to an article being assembled or a component of such an article, or to apparatus used to transport the web in a predetermined direction. The “configuration” of the web refers to the general condition of the web, such as whether the web is generally flat, folded or unfolded, wrinkled or unwrinkled, stretched or contracted, extended, etc.

As used herein, a “magnetically responsive material” applied to the material web refers to a material which is capable of reacting to the presence of a magnetic field, such as by being drawn toward or otherwise repelled by such a magnetic field. Desirably, the magnetically responsive material is selected from the group consisting of ferrimagnetic materials and superparamagnetic materials. A ferrimagnetic material is one in which the magnetic moments of neighboring domains tend to align anti-parallel to each other, but the moments are of different magnitudes. This is in contrast, for example, to a ferromagnetic material wherein the magnetic moments of neighboring domains tend to align in a common direction. Examples of ferrimagnetic materials include maghemite (gamma Fe₂O₃), magnetite (Fe₃O₄) and ferrite. Ferrite is a compound having the chemical formula XFe₂O₄, where X represents a divalent metal, such as iron or zinc, whose size is such that it will fit into the crystal's structure. However, it is contemplated that the magnetically responsive material may be a ferromagnetic material, such as iron, nickel, cobalt and various alloys of these materials, without departing from the scope of this invention.

Generally, the magnetically responsive material, and in particular the ferrimagnetic material when used as the magnetically responsive material applied to the web, has a coercivity of less than about 400 gauss (about 0.04 weber/square meter) and a remanence induction of less than about 2500 gauss (about 0.25 weber/square meter). Coercivity is the force required to demagnetize a material or, in other words, the amount of applied magnetic field that is required to overcome the magnetic induction of a material and bring its residual magnetisim back to zero. Remanence is the residual magnetic flux density that remains in a material after the removal of an applied magnetic field.

A superparamagnetic material is a material comprising fine particles in which the fine particles behave substantially independent of one another in the absence of a magnetic field but, when subjected to a magnetic field, are magnetized parallel to the magnetic field to an extent proportional to the magnetic field. One example of a superparamagnetic material is nanocrystals of gamma Fe₂O₃. The superparamagnetic material has a coercivity of about zero gauss and a remanence induction of about zero gauss. The magnetically responsive may alternatively be a diamagnetic material, such as bismuth or copper, which also remains unmagnetized in the absence of a magnetic field but tends to be repelled by a magnetic field in the presence thereof.

The magnetically responsive material is desirably particulate and is applied to the web in an amount sufficient to render the web magnetically responsive to the presence of a magnetic field but remain substantially unmagnetized in the absence of a magnetic field. As an example, the magnetically responsive material is desirably less than or equal to about 3 microns in size, and more desirably less than or equal to about 10 nanometers in size, and is applied to the material web in a concentration of less than about 20% by weight, and more desirably in the range of about 2% to about 8% by weight. It understood, however, that smaller or large sized particles may be used, and/or the concentration of magnetically responsive material may be other than as identified above, without departing from the scope of this invention. In one embodiment the magnetically responsive material is desirably generally elongate in shape. However, it is contemplated that the particles of magnetically responsive material may be of substantially any shape. It is also contemplated that the magnetically responsive material may be other than particulate, such as fibrous.

The magnetically responsive material is also desirably transparent, or at least translucent, so as to be generally invisible when the web to which the magnetically responsive material is applied is used or otherwise incorporated into an article such as a diaper, training pants, etc. The magnetically responsive material may optionally be white. For example, one suitable magnetically responsive material is a transparent, gamma Fe₂O₃ material developed by Xerox Corporation as reported in the Chemical & Engineering News Journal of Jul. 20, 1992. Alternatively, a colored magnetically responsive material may be coated or otherwise colored white, as long as the coating or other material used to color the material white does not substantially interfere with the magnetic properties of the magnetically responsive material. As an example, zinc oxide or cellulose acetate microfibers may be adhered to magnetically responsive material such as, for example, by using a fluidized bed process.

In one embodiment, the magnetically responsive material is applied to the material web by incorporating the magnetically responsive material into the web prior to formation thereof, such as by mixing the magnetically responsive material with the material from which the web is subsequently formed. In this manner, the magnetically responsive material becomes a chemical or physical part of, or is otherwise entrained in, the material web. Alternatively, the magnetically responsive material may be applied to the material web after formation thereof, such as by coating or spraying the magnetically responsive material onto an outer surface of the web. The magnetically responsive material may also be applied to the web in any other suitable manner, as long as there is no damaging affect on the magnetically responsive properties of the material and on the desired properties of the web itself. The magnetically responsive material may be applied to the material web generally throughout or over the entire web, or it may be applied to select regions of the web, such as toward the laterally opposite edge margins or longitudinal ends (e.g., where the web is a discrete web) thereof or along a lateral central region (e.g., down the center) of the web, so that only the select region or regions of the web to which the magnetically responsive material is applied are responsive to magnetic fields.

To further illustrate and describe the present invention, one embodiment of an apparatus and method for controlling a moving web is hereafter described in connection with making children's training pants, and more particularly for making disposable training pants as described in U.S. patent application Ser. No. 09/444,083 titled “Absorbent Articles With Refastenable Side Seams” and filed Nov. 22, 1999 (corresponding to PCT application WO 00/37009 published Jun. 29, 2000) by A. L. Fletcher et al., the disclosure of which is incorporated herein by reference to the extent it is consistent herewith. Training pants can also be constructed using the methods and apparatus disclosed in U.S. Pat. No. 4,940,464 issued Jul. 10, 1990 to Van Gompel et al.; and U.S. Pat. No. 5,766,389 issued Jun. 16, 1998 to Brandon et al.; the disclosures of which are also incorporated herein by reference to the extent they are consistent herewith.

It is to be understood, however, that the apparatus and methods described herein for controlling a moving web of material are applicable to substantially any apparatus in which a material web, whether continuous or discrete, is manipulated, such as by controlling the position, orientation and/or configuration thereof as the web is moved in a predetermined direction. For example, the embodiments shown and/or described herein are applicable to the manufacture of a variety of other garments or articles such as diapers, adult incontinence garments, medical garments, feminine care products, other personal care or health care garments; medical garments; swim pants; athletic clothing; pants and shorts and the like, or simply to manufacture continuous or discrete flexible webs of material which may be formed separate from garments or other articles and subsequently used either by itself or in the assembly of a garment or other article.

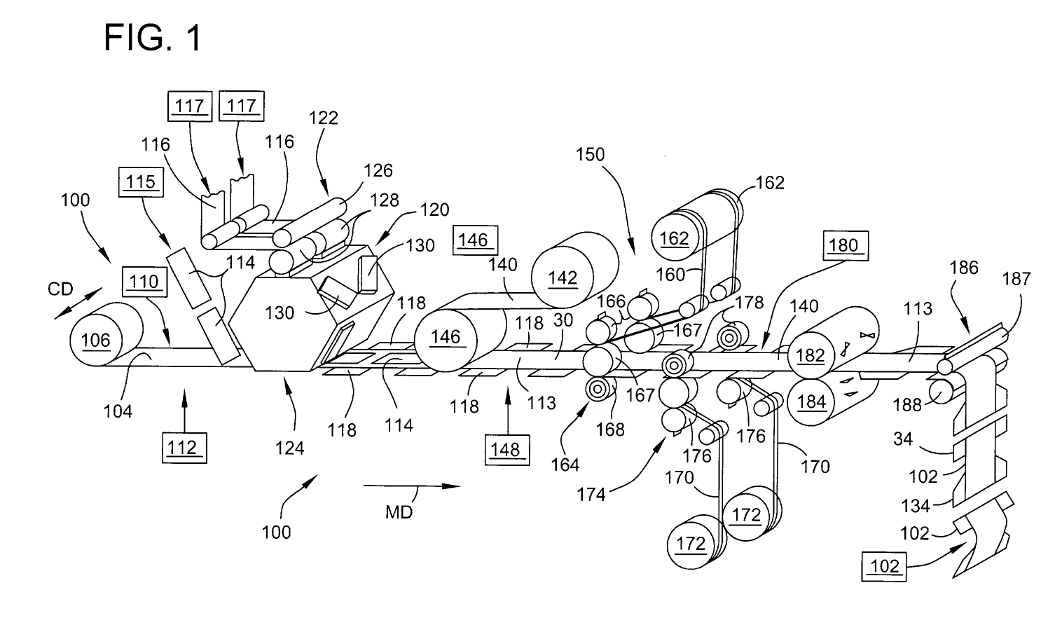

With reference now to the drawings, and in particular to FIG. 1, apparatus of the present invention for making a child's pair of training pants 102 is generally indicated at 100. The apparatus 100 comprises a continuous supply of a web material 104, which is used to form a bodyside liner of the pants 102, provided from a supply source 106. The supply source 106 can comprise for example any standard unwind mechanism (not shown), which generally includes a pair of spindles, a festoon assembly, and a dancer roll for providing the bodyside liner material web 104 at a desired speed and tension.

Various components of the training pants 102 can be disposed on and/or bonded to the bodyside liner material web 104 as it travels in a predetermined direction or path of movement, otherwise referred to herein as the machine direction MD. In the illustrated embodiment, the machine direction MD extends generally in a direction that the pants 102, or a particular component or material web thereof, is transported lengthwise along and through a particular, local position of the apparatus 100. A cross-machine direction CD of the apparatus 100 lies generally within the plane of the pants 102, or particular component or material web thereof, and is transverse or otherwise extends laterally relative to the machine direction MD.

A surge layer (not shown) can be provided at an application station 110 and disposed on and/or bonded to the bodyside liner material 104. The surge layer can comprise either a continuous web or discrete sheets. Additionally, a containment flap module 112 can be provided downstream from the supply source 106 for attaching pre-assembled containment flaps (not shown) to the bodyside liner material 104. As various components are added by the apparatus 100, a continuously moving product assemblage 113 is formed and moved generally in the machine direction MD, or direction of assembly. The product assemblage 113 will be cut downstream to form partially assembled, discrete training pants 102.

Absorbent assemblies 114 can be provided from a suitable supply source 115, which can be any conventional mechanism for supplying the absorbent assemblies 114. Generally, a conventional supply source 115 can include a hammermill for forming fluff fibers and, if desired, for providing an enclosure for mixing superabsorbent material with the fluff fibers, and then depositing the fluff and superabsorbent material on a forming drum having a desired absorbent design. The individual absorbent assemblies 114 can be disposed intermittently on the continuously moving bodyside liner material 104, one for each pair of training pants 102 to be assembled. The position of the absorbent assemblies 114 can be registered with the position of the surge material, if employed. The absorbent assemblies 114 can be bonded to one or more other components using adhesives or other suitable means. Alternatively, composite absorbent materials can be fed into the converting process from rolls or compressed packages, such as festooned bales.

A pair of continuous webs of material 116 used to form front and back side panels 34 and 134 of the pants (e.g., wherein each material web corresponds to a respective one of the laterally opposite sides of the pants 102 to be assembled) can be provided from suitable supply sources 117, such as standard unwind mechanisms. The side panel material webs 116 desirably have a magnetically responsive material applied thereto as described previously. For example, in one embodiment the magnetically responsive material is desirably applied to the side panel material webs 116 prior to the webs being rolled and stored for subsequent mounting on the supply sources 117, and more desirably the magnetically responsive material is incorporated into the side panel material webs prior to formation of the webs. Alternatively, the magnetically responsive material may be applied to all or part of each web 116 by a suitable spray coating or other applicating device (not shown) as the webs are unwound from the supply sources 117. The supply sources 117 can comprise one or more standard unwind mechanisms (not shown).

The side panel material webs 116 can be cut into discrete strips 118 and positioned partially on the bodyside liner material 104 using an applicator device, generally indicated at 120. The individual strips 118 desirably extend laterally out (e.g., in the cross-machine direction CD) from the bodyside liner material web 104 and overlap the bodyside liner material web, for example, by an amount such as about 2 or more centimeters, to permit bonding of the strips to the bodyside liner and/or the containment flap material. In the machine direction MD, the position of the strips 118 can be registered relative to the absorbent assemblies 114 so that the product assemblage 113 can be cut between the absorbent assemblies with each strip 118 of side panel material 116 forming both a front side panel 34 and a back side panel 134 of consecutive pants 102.

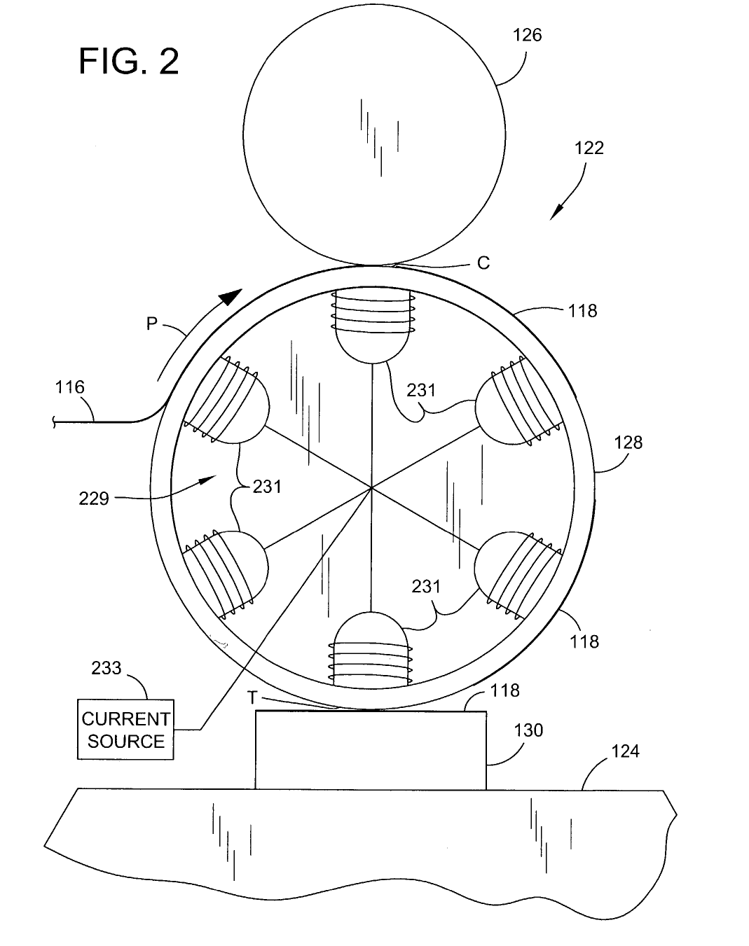

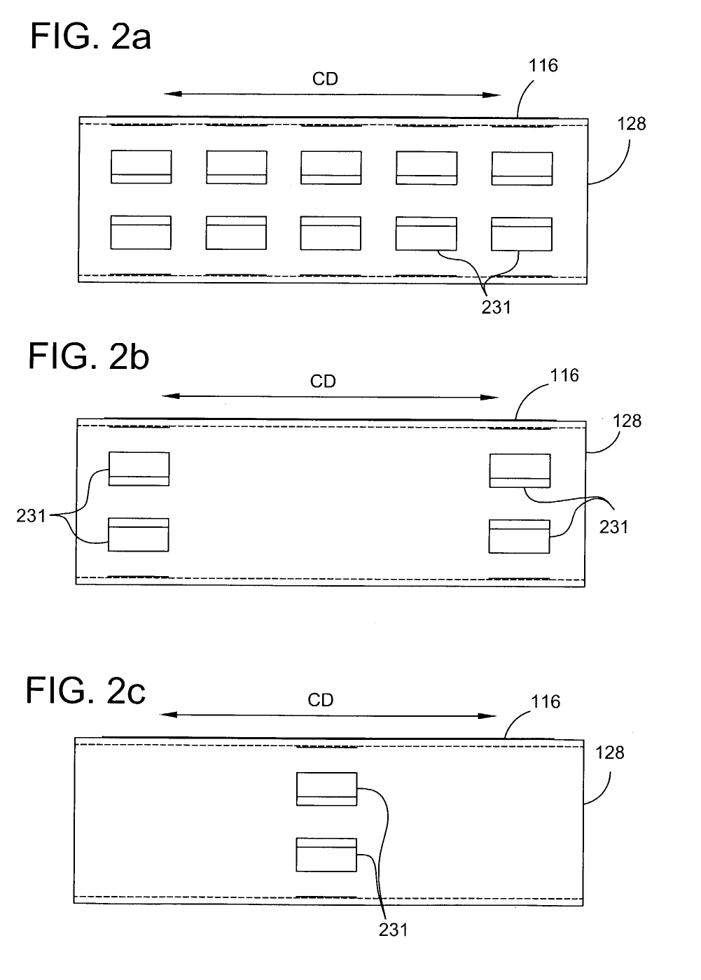

The applicator device 120 of the illustrated embodiment comprises a cutting assembly, generally indicated at 122, for cutting the side panel material webs 116 into discrete strips 118 of material webs, and a rotatable transfer roll, generally indicated at 124, for transferring the discrete strips onto the continuously moving bodyside liner material web 104. The cutting assembly 122 comprises a rotatable knife roll 126 and rotatable anvil rolls 128 (e.g., each corresponding to a respective one of the side panel material webs 116) arranged end-to-end with each other in opposed relationship with the knife roll. However, it is contemplated that a single anvil roll (not shown) may be used instead of two separate anvil rolls 128. With reference to FIG. 2, each anvil roll 128 (broadly, a web support) comprises a magnetic field generator, generally indicated at 229, operable to generate a magnetic field which attracts the magnetically responsive material applied to the side panel material web 116 to thereby urge the web toward the outer surface of the anvil roll into contiguous relationship therewith (e.g., in contact or closely spaced relationship therewith) as the material web is cut into discrete strips 118 by the knife roll 126 and then rotated into position for transfer to the transfer roll 124.

In the embodiment illustrated in FIG. 2, the magnetic field generator 229 comprises at least one and more particularly a plurality of electromagnets, which are illustrated schematically in FIG. 2 and indicated at 231, disposed on or within the interior of the anvil roll 128 in spaced relationship with each other about the circumference of the anvil roll. The electromagnets 231 are also spaced longitudinally along the length of the anvil roll 128 (e.g., in the cross-machine direction CD) as shown in FIG. 2a so that the magnetic fields generated by the electromagnets are present along the length of the anvil roll. Alternatively, the electromagnets 231 spaced circumferentially about the anvil roll 128 may be elongate (not shown) and extend longitudinally along the length of the anvil roll 128. It is also contemplated that the electromagments 231 may extend (e.g., continuously or in spaced relationship with each other) only along a discrete portion, or portions, of the anvil roll length. For example, electromagnets 231 may be disposed only toward the ends of the anvil roll 128 as shown in FIG. 2b, only at the center of the anvil roll as shown in FIG. 2c, or in another desired pattern without departing from the scope of this invention.

In the illustrated embodiment, the electromagnets 231 are connected to the anvil roll 128 for conjoint movement with the anvil roll upon rotation with the anvil roll. More particularly, the electromagnets 231 are embedded within or otherwise secured to the anvil roll 128 with the electromagnet generally flush with the outer surface of the anvil as shown in FIG. 2a to thereby at least partially form the outer surface of the anvil roll. The remainder of the anvil roll is desirably constructed of a non-ferrous material such as, for example, aluminum, carbon fiber composite or another suitable non-ferrous material. Alternatively, the anvil roll may constructed entirely of a ferrous material and remain within the scope of this invention.

It is also contemplated that the electromagnets 231 may instead be connected (not shown) to the inner surface of the anvil roll, e.g., without forming part of the outer surface of the anvil roll. In such an embodiment, the anvil roll is desirably constructed of a non-ferrous material to allow the magnetic field generated by the electromagnets 231 to pass through the anvil roll for attracting the material web 116 to the outer surface of the anvil roll.

The electromagnets 231 are in electrical communication with a source 233 of electrical current whereby the electromagnets generate a magnetic field when current is supplied to the electromagnets. It is contemplated that each of the electromagnets 231 may instead be in electrical communication with its own individual source of current, or that two or more groupings of electromagnets 231 may each be in electrical communication with a respective source of current. It is also contemplated that a suitable control (not shown) may be electrically intermediate the source of current and the electromagnets 231 to control the amount of current delivered to each of the electromagnets, and that each electromagnet may have a different amount of current delivered thereto, without departing from the scope of this invention. Such a control or controls may also, or may instead, selectively deliver current to and then inhibit the delivery of current to the respective electromagnet(s) to which the control is electrically connected.

With each revolution of the anvil roll 128, the outer surface of the anvil roll follows a rotational path (indicated by the arrow P in FIG. 2) including a cutting position C (broadly, a first position) wherein the anvil roll outer surface is in opposed relationship with the knife roll and a subsequent transfer position T (broadly, a second position) wherein the anvil roll outer surface is in opposed relationship with the transfer roll 124. As the continuous side panel material web 116 passes from the supply source 117 over the anvil roll 128 at the cutting position C thereof, electrical current is supplied to the electromagnets 231 secured to the anvil roll generally at the cutting position of the anvil roll to urge the web toward the outer surface of the anvil roll as the web is cut into the strips 118.

The strips 118 are held by the magnetic field in contiguous relationship with the outer surface of the anvil roll 128 upon rotation thereof until the strips approach the transfer position T. The magnetic field holding the strips against the anvil roll is then desirably disrupted so that the magnetic field generated by the electromagnets 231 approaching the transfer position T is weaker, fully dissipated or even reversed relative to the magnetic field generated by the electromagnets at the cutting position C. For example, the magnetic field generated by the electromagnets 231 may be disrupted by reducing the current flow to the electromagnets, by interrupting the current flow to the electromagnets or by reversing the current flow through the electromagnets. The disruption may be a stepped, e.g., instant disruption or it may be a gradual disruption that occurs as the anvil roll rotates the strips 118 from the cutting position C toward the transfer position T. Providing a disrupted magnetic field at the transfer position T facilitates the release of the strips 118 from the anvil roll 128 for transfer onto the transfer roll 124. Transfer of the strips 118 from the anvil roll 128 may also be assisted by additional devices, such as air blowers or other suitable devices, and remain within the scope of this invention.

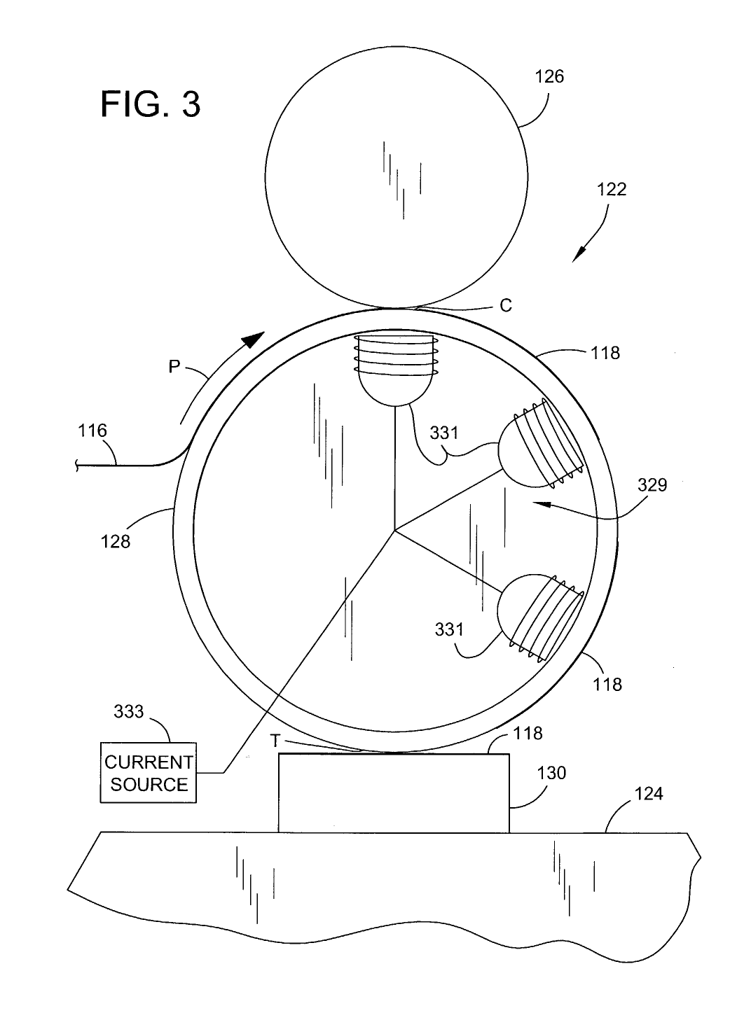

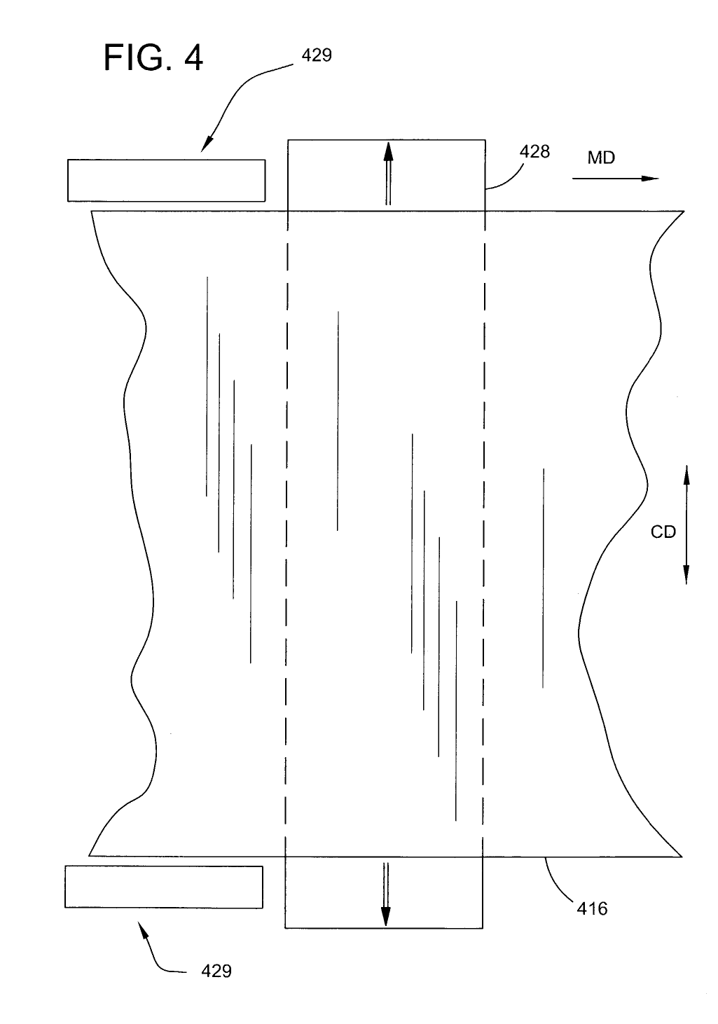

In another embodiment, the electromagnets may be fixed within the anvil roll against movement therewith such that the anvil roll rotates relative to the electromagnets. For example, as shown in FIG. 3, electromagnets 331 are disposed in circumferentially spaced relationship with each other about only a portion of the circumference of the anvil roll 128. It is contemplated that the electromagnets 331 may be disposed at two or more discrete portions, or zones, about the circumference of the anvil roll 128. The electromagnets 331 are also disposed longitudinally along all, or one or more discrete portions, of the length of the anvil roll 128 as described previously with respect to the electromagnets 231 of FIGS. 2a, 2b and 2c. In this embodiment, electrical current is supplied to the electromagnets 331 to generate a magnetic field generally along the rotational path P of the outer surface of the anvil roll 128 between the cutting position C and the transfer position T of the anvil roll. As the anvil roll 128 rotates past the electromagnets 331 at the cutting position C of the anvil roll, the magnetic field generated by the electromagnets attracts and urges the material web 116 toward the anvil roll outer surface as the web is cut into strips 118 and then rotates the strips toward the transfer position T of the anvil roll.

The magnetic field holding the strips 118 in contiguous relationship with the anvil roll 128 is desirably disrupted as the strips approach the transfer position T such that the magnetic field generated by the electromagnets 331 nearer the transfer position T is weaker, fully dissipated or even reversed relative to the magnetic field generated by the electromagnets at the cutting position C. For example, the magnetic field generated by the electromagnets 331 nearer the transfer position T may be disrupted by delivering a weaker current flow to those electromagnets than the current flow delivered to the electromagnets at the cutting position C, by intermittently interrupting the current flow to those electromagnets or by reversing the current flow through those electromagnets. The disruption may be a stepped, e.g., instant disruption or it may be a gradual disruption that occurs as the anvil roll rotates the strips 118 past circumferentially successive electromagnets from the cutting position C toward the transfer position T. That is, the magnetic field strength may decrease at successive electromagnets 331 along the rotation path of the anvil roll 128.

Providing a disrupted magnetic field at the transfer position T facilitates the release of the strips 118 from the anvil roll 128 for transfer onto the transfer roll 124. In the illustrated embodiment of FIG. 3, the disruption of the magnetic field occurs as a result of the absence of an electromagnet 331 at the transfer position T. However, it is understood that an electromagnet 331 may be positioned generally at the transfer position and remain within the scope of this invention.

It is understood that the magnetic field generator 229, 329 of the anvil roll 128 may instead comprise one or more permanent magnets (not shown) or other devices suitable for generating a magnetic field without departing from the scope of this invention. In such an embodiment, a disrupted magnetic field may occur as a result of an absence of a permanent magnet, or a permanent magnet within the anvil roll may be selectively moved toward or away from the anvil roll outer surface to control the strength of the magnetic field to which the web 116 is exposed. It is also contemplated that the anvil roll 128 may instead be constructed, in whole or in part, of a ferrous material and have an electrically conductive coil extending longitudinally within the anvil roll in electrical communication with a source of electrical current whereby supplying the coil with current magnetizes the entire outer surface of the anvil roll (e.g., in the manner of an electromagnet) to attract the magnetically responsive material applied to the material web 116.

Referring back to FIG. 1, the rotatable transfer roll 124 comprises one or more pucks 130 (broadly, web supports), wherein each puck comprises a magnetic field generator (not shown but substantially similar to either of the magnetic field generators 229, 329 of the anvil roll 128 shown in FIGS. 2 and 3) operable to generate a magnetic field which attracts the magnetically responsive material applied to the web material 116 to thereby urge the strips 118 toward the outer surface of the pucks 130. Upon rotation of the transfer roll 124, the pucks 130 rotate through an angle of about 90 degrees relative to the transfer roll and to the continuously moving bodyside liner material web 104 to change the angular orientation of the strips relative to the bodyside liner material web and to position the strips thereon. Alternatively, if reorientation of the strips 118 is not necessary, the transfer roll 124 may be a cylinder (e.g., without the need for the pucks 130) having a magnetic field generator (not shown) similar to the anvil roll 128 as described above.

In one embodiment, the magnetic field generator of each puck comprises one or more electromagnets (not shown but similar to the electromagnets shown in FIGS. 2 and 3) disposed within the puck and in electrical communication with a source (not shown) of current. Upon rotation of the strips 118 from the cutting position C of the anvil roll 128 to the transfer position T thereof, current is supplied to the electromagnets of the pucks 130 to generate a magnetic field which urges the strips toward the pucks 130 as the transfer roll and pucks are rotated away from the anvil roll. For example, the magnetic field urging the strips 118 toward the pucks 130 is desirably stronger than the magnetic field urging the strips toward the anvil roll at the transfer position T to draw the strips away from the anvil roll and toward the transfer roll pucks.

When the strips 118 are subsequently positioned and oriented by the transfer roll 124 and pucks 130 as desired relative to the bodyside liner material web 104, the magnetic field urging the strips toward the pucks is disrupted, such as in the manner described above with respect to the anvil roll 128 at the transfer position T thereof. A weaker, or even dissipated or reversed magnetic field facilitates releasing of the strips 118 from the transfer roll 124 onto the bodyside liner material web 104. The pucks 130 can continue to rotate relative to the transfer roll 124 as the transfer roll rotates back toward the cutting assembly 122 to receive other strips 118.

Alternatively, the magnetic field generator associated with each puck 130 may comprise a permanent magnet or other suitable device capable of generating a magnetic field without departing from the scope of this invention.

A continuous supply of another material web 140, used to form an outer cover of the training pants 102, is provided from a supply roll 142 or other suitable source. The outer cover material web 140 can be transported over a laminator roll 144 and bonded to the bodyside liner material web 104. The absorbent assemblies 114 are thereby sandwiched between the continuous material webs 104 and 140. The inward portions of the strips 118 of side panel material 116 can also be disposed between the bodyside liner material 104 and the outer cover material 140. Alternative configurations for attaching the side panel material webs 116 are disclosed by Van Gompel et al. Various components such as leg elastics (not shown) or waist elastics (not shown) can be bonded to the outer cover material 140 at an application station 146 prior to uniting the bodyside liner and outer cover material webs 104 and 140. Alternatively, leg elastics or waist elastics can be initially bonded to the bodyside liner material web 104 or another material.

Bonding devices 148 such as ultrasonic bonders can be employed downstream from the laminator roll 144 to bond the bodyside liner material web 104, side panel material web 116 and outer cover material web 140. For example, these material webs can be transported between a rotary ultrasonic horn and an anvil roll. Suitable rotary ultrasonic horns are described in U.S. Pat. No. 5,110,403 to Ehlert, which is incorporated herein by reference. Such rotary ultrasonic horns generally have a diameter of from about 5 to about 20 centimeters and a width of from about 2 to about 15 centimeters. Alternatively, the ultrasonic horn may be a stationary ultrasonic horn as is also known to those skilled in the art. Other suitable ultrasonic horns and ultrasonic bonders are commercially available from Branson Sonic Power Company, Danbury, Conn., U.S.A. The bonding devices 148 could otherwise be a thermal or adhesive bonder as are well known.

The continuously moving product assemblage 113 next advances to a fastener application station 150 where fastening components are bonded to the strips 118 of side panel material web 116. The location of the fastening components on the pants 102 is a function in part of the configuration of the apparatus 100. In the illustrated embodiment, the apparatus 100 is configured so that the upward facing surface of the product assemblage 113 will become the outer surface of the training pants 102 and the downward facing surface will become the inner surface.

Moreover, the illustrated apparatus 100 is configured to produce partially assembled training pants 102 having one longitudinal end of a leading pair of pants connected to an opposed longitudinal end of a trailing pair of pants. However, it is understood that the apparatus 100 could alternatively employ any combination of different orientations. For example, the upward facing surface of the product assemblage 113 could form the inner surface of the pants 102. Still alternatively, the apparatus 100 could be constructed as a cross-machine direction CD process wherein a longitudinal axis of each pair of pants 102 could be perpendicular to the machine direction MD during part or all of the assembly process.

With reference again to FIG. 1, continuous material webs 160 used to form the second fastening components can be provided from supply rolls 162 or other suitable sources. The second fastener material webs 160 can be cut into individual fastening components by cutting assemblies 164 or other suitable devices. The illustrated cutting assemblies 164 include rotatable knife rolls 166, rotatable vacuum anvil rolls 167, and rotatable backing rolls 168. The continuous second fastener material webs 160 can be cut by blades on the knife rolls 166, maintained on the anvil rolls 167 by vacuum, and disposed on the top surfaces of the strips 118 of side panel material webs 116.

Similarly, continuous material webs 170 used to form the first fastening components can be provided from supply rolls 172 or other suitable sources. The first fastener material webs 170 can be cut into individual fastening components by cutting assemblies 174 or other suitable devices. The illustrated cutting assemblies 174 include rotatable knife rolls 176, rotatable vacuum anvil rolls 177, and rotatable backing rolls 178. The continuous first fastener material webs 170 can be cut by blades on the knife rolls 176, maintained on the anvil rolls 177 by vacuum, and disposed on the undersides of the strips 118 of side panel material webs 116.

It is contemplated that other arrangements can be used to attach the fastening components. For example, the fastening components can be applied to the side panel material webs 116 prior to uniting the side panel material webs with the bodyside liner material web 104 and/or the outer cover material web 140; the fastening components can be applied to the bodyside liner material web 104 and/or outer cover material web 140, whether separate side panels 34, 134 are used or not; portions of other components such as the bodyside liner and/or outer cover can form one or more of the fastening components; the separate side panels or integral side panels can themselves form one or more of the fastening components; the fastening components can be attached as pre-engaged composites; or the like without departing from the scope of this invention.

After the fastening components are disposed on the strips 118 of side panel material webs 116, bonding devices 180 such as ultrasonic bonders can be employed to bond the fastening components to the strips. For example, the strips 118 can be transported between a rotary ultrasonic horn and an anvil roll, which devices are positioned on each side of the process at the cross machine direction location of the fastening components. Particular ultrasonic bond patterns comprising individual, circular bonds which are compatible with mechanical fastening materials are disclosed in U.S. Pat. No. 5,660,666 issued Aug. 26, 1997 to Dilnik et al., which is incorporated herein by reference. Efficient arrangements for attaching the fastening components with nonadhesive bonding devices are further described in published U.S. Patent Application No. US2002-0000291, filed on May 15, 2001 by J. D. Coenen et al. and titled “Methods For Making Garments With Fastening Components,” which is incorporated herein by reference. For secure attachment, it may be desirable to attach the fastening components with both adhesive and thermal bonds. Suitable attachment adhesives are available from commercial vendors such as Findley Adhesive, Inc., Wauwatosa, Wisconsin, U.S.A.

The strips 118 of side panel material webs 116 can be trimmed if desired, for example to provide angled and/or curved side panels 34, 134, particularly at the leg openings of the pants 102. To this end, the apparatus 100 can include a die cutting roll 182 and a backing roll 184.

The apparatus 100 to this point provides a continuous assemblage 113 of interconnected and partially assembled pairs of training pants moving in the machine direction MD. This continuously moving product assemblage 113 is passed through a cutter 186 which selectively cuts the web into discrete, partially assembled training pants 102. Such cutters 186 are generally known to those skilled in the art and can include, for example, the combination of a cutting roll 187 and an anvil roll 188 through which the web travels. The anvil roll 188 can include a hardened steel rotating roll while the cutting roll 187 can include one or more flexible hardened steel blades clamped onto another rotating roll. The pinching force between the blade on the cutting roll 187 and the anvil roll 188 creates the cut. The cutting roll 187 can have one or more blades depending upon the desired distance between the cuts. The cutter 186 can further be configured to provide a spacing between the individual cut pieces after they are cut. Such a spacing can be provided by transferring the cut pieces away from the cutter at a higher speed than the speed at which the web is provided to the cutter.

While magnetic control of a moving material web is described above in connection with the side panel material web 116, the anvil roll 128 comprising the magnetic field generator 229, 329, and the pucks 130 of the rotatable transfer roll 124 also comprising a magnetic field generator, it is understood that other material webs used in making the pants 102 may have a magnetically responsive material applied thereto, and/or other components of the apparatus 100 of FIG. 1 may comprise a magnetic field generator for controlling the one or more material webs to which a magnetically responsive material is applied. For example, the fastener material webs 160, 170 can have a magnetically responsive material applied thereto. The vacuum anvil rolls 167, 177 may be replaced with anvil rolls (broadly, web supports) comprising respective magnetic field generators similar to the magnetic field generator 229 of FIG. 2 or the magnetic field generator 329 of FIG. 3.

Also, one or both of the bodyside liner material web 104 and the outer cover material web 140 can have a magnetically responsive material applied thereto. In such an embodiment, the anvil roll 188 (broadly, a web support) of the cutter 186 may comprise a magnetic field generator (not shown but similar to the magnetic field generator 229 of FIG. 2 or the magnetic field generator 329 of FIG. 3) which generates a magnetic field to urge the moving product assemblage 113 toward the outer surface of the anvil roll as the assemblage is cut by the cutter 187 into discrete training pants 102. It is also contemplated that the bodyside liner material web 104 may be supported by a conveyor (broadly, a web support, not shown) which moves the web in the machine direction MD wherein the conveyor has one or more magnetic field generators which generate a magnetic field to urge the bodyside liner material web, and hence the product assemblage 113 formed on the web, toward the conveyor. Alternatively, one or more idler rolls (also broadly referred to as a web support, not shown) may support the bodyside line material web 104 upon movement of the web in the machine direction. It is contemplated that such an idler roll can be constructed similar to the anvil roll 128 of FIG. 3 whereby a magnetic field is generated over only that portion of the idler roll which is intended to support the web.

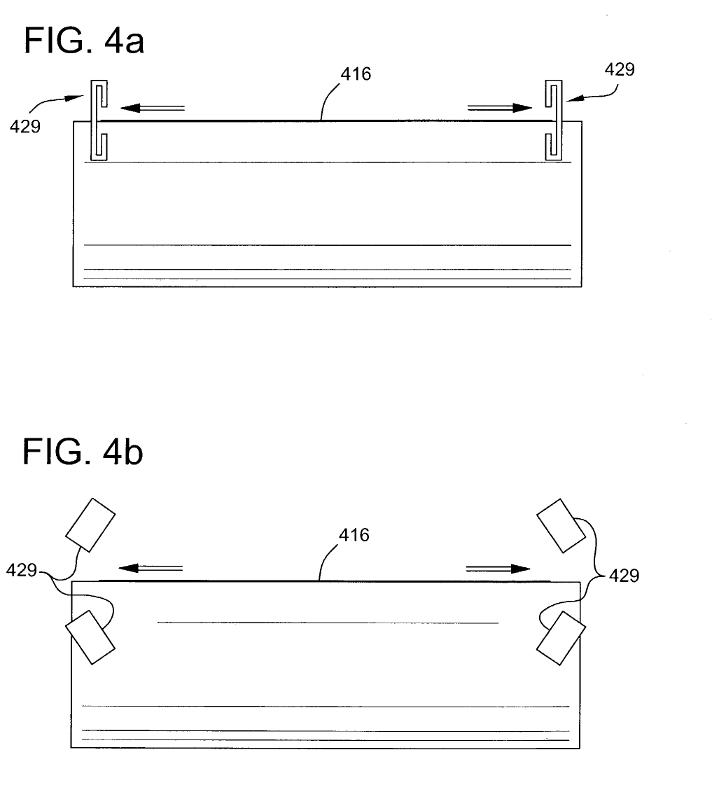

It is also contemplated that a variety of other manipulations may be performed on a moving web having a magnetically responsive material applied thereto. For instance, in other embodiments one or more magnetic field generators may be used to configure the moving web in a desired configuration, such as by folding or unfolding the web, laying the web flat, wrinkling or unwrinkling the web, extending or stretching the web, etc. As an example, a pair of magnetic field generators, generally indicated at 429, are shown in FIG. 4 as being positioned upstream of a roll 428 (broadly, a web support) over which a continuous web of material 416, having a magnetically responsive material applied thereto as described previously with respect to the side panel material web 116, passes in contiguous relationship (e.g., in contact or closely spaced relationship) therewith upon movement of the web in a predetermined direction. The web 416 may rotate with the roll 428 through a portion of one revolution thereof, or the web may slip relative to the surface of the roll while remaining in contiguous relationship therewith along a portion of the outer circumference of the roll. The magnetic field generators 429 are spaced laterally (e.g., in the cross-machine direction CD) from each other and positioned generally at the laterally opposite side edges of the material web 416.

For example, as shown in FIG. 4a, each magnetic field generator 429 may be generally C-shaped and be positioned at (e.g., in generally opposed and/or laterally adjacent relationship with) a side edge of the material web 416 to surround the side edge with the generator positioned at least partially laterally outward of the side edge of the material web. The magnetic field generators 429 are thus operable to generate opposed magnetic fields generally at the side edges of the web to urge the side edges of the moving web 416 in laterally opposite directions as indicated by the force arrows shown in FIG. 4 to thereby configure the web in a generally taut configuration. Tensioning the web 416 in this manner tends to configure the web in a generally unwrinkled configuration as the web moves into contiguous relationship with the roll 428 upon movement in the predetermined direction.

In another embodiment illustrated in FIG. 4b, a pair of magnetic generators 429 are positioned at each side edge of the moving material web 416 with one generator disposed above the web and other generator disposed below the web. The generators are oriented to generate magnetic fields which have both a lateral and a normal (e.g., to the plane of the web 416) component whereby the lateral components of the magnetic fields generated at the opposite side edges of the web urge (e.g., pull) the side edges of the moving web in laterally opposite directions to thereby configure the web in a generally taut configuration.

Alternatively, as described previously and shown in FIG. 2b, magnetic field generators 229 may be disposed within the roll 128 generally adjacent the ends of the roll, or at least partially outward of the side edges of the material web 116 when the web is in contiguous relationship with the outer surface of the roll, so that magnetic fields are generated only toward the outer ends (e.g., in the cross-machine direction CD) of the roll. As the material web 116 moves in the predetermined direction in contiguous relationship with the roll, the side edges of the web are urged outward by the magnetic fields to facilitate the web being pulled taut to thereby unwrinkle the web.

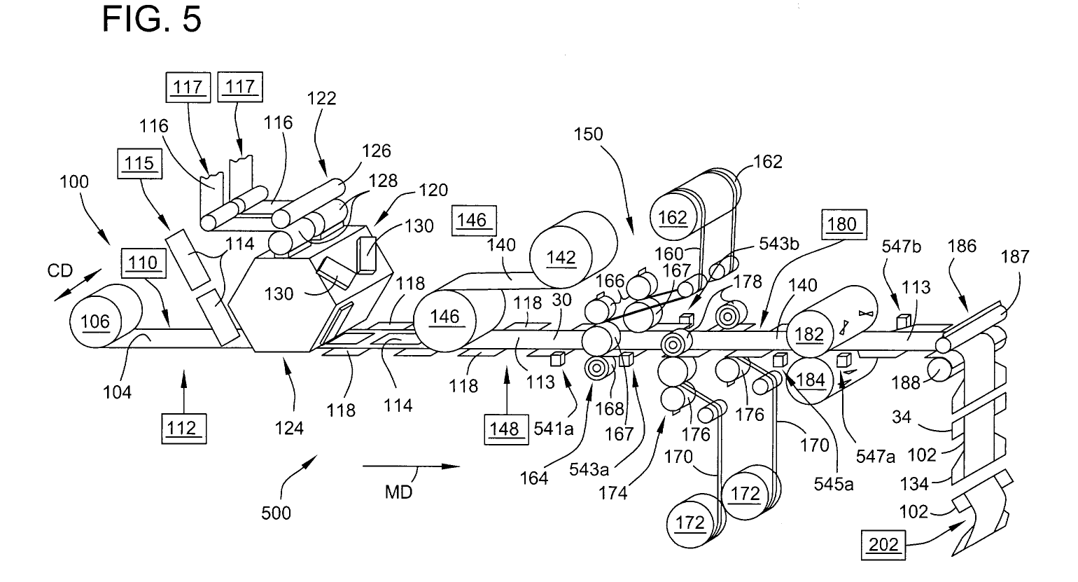

One or more magnetic field generators can also be used to configure a moving material web in a generally extended configuration, or even a stretched configuration if the web material is stretchable. For instance, FIG. 5 illustrates an apparatus 500 similar to the apparatus 100 of FIG. 1 for making training pants. Following securement of the strips 118 of side panel material 116 to the product assemblage 113, the continuous product assemblage may be transported past first and second pairs of laterally opposite magnetic field generators (with only one such magnetic field generator of a first pair being shown schematically in FIG. 5 and indicated generally at 541a and a second pair being shown schematically and indicated generally at 543a and 543b), e.g., positioned on laterally opposite sides of the product assemblage generally at the respective machine direction MD locations at which the fastening component material webs 160, 170 are secured to the strips.

The magnetic fields generated by the magnetic field generators 541a, 543a, 543b attract the magnetically responsive material of the side panel material web 116 to generally urge the side panel strips 118 outward relative to the product assemblage 113 (e.g., and to a longitudinal axis of each strip) to an extended configuration of each strip to facilitate proper positioning of the fastening components on the side panel strips. A similar pair of laterally opposed magnetic field generators, one of which is shown schematically in FIG. 5 and indicated generally at 545a, may be located just upstream of the die cutting roll 182 and backing roll 184 to configure the side panel strips 118 in an extended configuration to facilitate proper cutting of the strips to define the cut side panels 34, 134 of the pants 102. It is also contemplated that laterally opposite collection devices, shown schematically in FIG. 5 and indicated generally at 547a, 547b, may disposed on the downstream side of the die cutting roll 182 and backing roll 184 to collect the cut portions of the side panel material webs 116 and to transfer the cut portions to a suitable collection bin (not shown). Each collection device comprises a magnetic field generator operable to generate a magnetic field which attracts and collects the cut portions of the side panel material webs 116.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. For example, features described in relation to one embodiment may be incorporated into any other embodiment of the invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

When introducing elements of the present invention or the preferred embodiment(s) thereof, the articles a, an, the and said are intended to mean that there are one or more of the elements. The terms comprising, including and having are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A method of controlling at least one of the position, the orientation and the configuration of a material web during movement of said web in a predetermined direction, said method comprising the steps of:
applying a magnetically responsive material to the material web; and
exposing the material web to at least one magnetic field during movement of the web in said predetermined direction, said at least one magnetic field attracting or repelling the magnetically responsive material applied to the material web to thereby control at least one of the position, the orientation and the configuration of said web during movement of the web in said predetermined direction.

2. A method as set forth in claim 1 wherein the step of exposing the material web to at least one magnetic field comprises moving said web in the predetermined direction past at least one magnetic field generator and operating said at least one magnetic field generator to generate a magnetic field in the presence of the magnetically responsive material applied to the web.

3. A method as set forth in claim 2 wherein the step of exposing the material web to a magnetic field further comprises moving the web into contiguous relationship with a web support upon movement of the material web in said predetermined direction, the web support comprising a magnetic field generator, and operating the magnetic field generator to generate a magnetic field in the presence of the magnetically responsive material applied to the web to urge the material web toward the web support as the web is moved in said predetermined direction.

4. A method as set forth in claim 3 wherein the web support comprises a roll rotatable along a rotational path relative to the predetermined direction of movement of the material web, the roll having an outer surface and comprising a magnetic field generator, the step of exposing the material web to the magnetic field comprising rotating the roll and moving the material web into contiguous relationship with the outer surface of the roll upon movement of the web in said predetermined direction, and operating the magnetic field generator to generate a magnetic field to urge the material web toward the outer surface of the roll to move along at least a portion of the rotational path of the roll as the material web is moved in the predetermined direction.

5. A method as set forth in claim 4 wherein the roll is rotatable through a rotational path including a first position along said rotational path and a second position along said rotational path different from said first position, the step of exposing the material web to a magnetic field comprising exposing the material web to a first magnetic field sufficient to urge the material web toward the outer surface of the roll generally at said first position, and further comprising disrupting the magnetic field at the second position to facilitate the release of the material web from the roll.

6. A method as set forth in claim 5 wherein the step of disrupting the magnetic field at said second position comprises one of dissipating the magnetic field at said second position, decreasing the strength of the magnetic field at said second position relative to the strength of the magnetic field at said first position, and reversing the magnetic field at said second position relative to the magnetic field at said first position, to facilitate release of the material web from the roll.

7. A method as set forth in claim 1 wherein the magnetic field configures the material web in at least one of an unwrinkled configuration, an extended configuration and a stretched configuration.

8. A method as set forth in claim 1 wherein the material web has a longitudinal axis, a lateral axis and laterally opposite side edges, the step of exposing the material web to at least one magnetic field comprising exposing at least one of said side edges of the material web to a magnetic field disposed generally at at least one side edge of the material web whereby the magnetic field has a lateral component which urges the web in a direction generally parallel to the lateral axis of said web.

9. A method as set forth in claim 8 wherein the step of exposing the material web to at least one magnetic field comprises exposing the laterally opposite side edges of the material web to laterally opposed magnetic fields, each of the magnetic fields being disposed at a respective one of the side edges of the material web whereby the opposed magnetic fields have respective lateral components which urge the side edges of the web in laterally opposite directions to thereby configure the magnetic web in a generally taut configuration.

10. A method as set forth in claim 9 wherein the step of exposing the laterally opposite side edges of the material to laterally opposed magnetic fields comprises moving the material web in said predetermined direction past a pair of laterally opposed magnetic field generators, each magnetic field generator being positioned generally at a respective one of the side edges of the material web.

11. A method as set forth in claim 1 wherein the material web is a generally continuous material web.

12. A method as set forth in claim 1 wherein the web is constructed of a web material, the step of applying the magnetically responsive material to the web comprising mixing the magnetically responsive material with the web material and then forming the web.

13. A method as set forth in claim 1 wherein the web is constructed of a web material, the step of applying the magnetically responsive material to the web comprising applying the magnetically responsive material to an outer surface of the material web.

14. A method as set forth in claim 1 wherein the magnetically responsive material is selected from the group comprising ferrimagnetic materials, superparamagnetic materials and ferromagnetic materials.

15. A method as set forth in claim 14 wherein the magnetically responsive material is selected from the group comprising maghemite, magnetite, ferrite and nanocrystals of gamma Fe₂O₃.

16. A disposable article comprising at least one material web, said material web having a magnetically responsive material applied thereto.

17. A disposable article as set forth in claim 16 herein said disposable article is one of a diaper, a pair of training pants, a feminine care product, an adult incontinence product and a medical garment.

18. A disposable article as set forth in claim 16 wherein the disposable article comprises a plurality of material webs held in assembly with each other, at least one of which has a magnetically responsive material applied thereto.

19. A disposable article as set forth in claim 16 wherein said disposable article comprises a bodyside liner material web, an outer cover material web, and an absorbent assembly disposed between the liner material web and the outer cover material web, at least one of the bodyside liner material web and the outer cover material web having a magnetically responsive material applied thereto.

20. A disposable article as set forth in claim 16 wherein said disposable article comprises a bodyside liner material web, an outer cover material web, an absorbent assembly disposed between the liner material web and the outer cover material web, and at least one other material web different from the bodyside liner material web and the outer cover material web, at least one of the bodyside liner material web, the outer cover material web and said at least one other material web having a magnetically responsive material applied thereto.

21. An absorbent article as set forth in claim 20 wherein the absorbent article is a pair of training pants, said at least one other material web comprising at least one pair of laterally opposite side panel material webs disposed on laterally opposite sides of the article.

22. A disposable article as set forth in claim 16 wherein the magnetically responsive material is selected from the group comprising ferrimagnetic materials, superparamagnetic materials and ferromagnetic materials.

23. A disposable article as set forth in claim 16 wherein the magnetically responsive material is selected from the group comprising maghemite, magnetite, ferrite and nanocrystals of gamma Fe₂O₃.

24. A disposable article as set forth in claim 16 wherein the magnetically responsive material comprises particles of magnetically responsive material having a particle size of less than or equal to about 3 microns.

25. A disposable article as set forth in claim 24 wherein the magnetically responsive material comprises particles of magnetically responsive material having a particle size of less than or equal to about 10 nanometers.

26. A disposable article as set forth in claim 16 wherein the concentration of magnetically responsive material applied to the at least one material web is less than about 20 percent by weight.

27. A disposable article as set forth in claim 26 wherein the concentration of magnetically responsive material applied to the at least one material web is in the range of about 2 percent to about 8 percent by weight.

28. Apparatus for magnetically controlling at least one of the position, the orientation and the configuration of a material web upon movement of the web in a predetermined direction, said web having a magnetically responsive material applied thereto, said apparatus comprising:
at least one magnetic field generator operable to generate a magnetic field in magnetically responsive communication with the material web upon movement of the web in said predetermined direction, said magnetic field being capable of one of attracting and repelling the magnetically responsive material applied to the material web.

29. Apparatus as set forth in claim 28 wherein the at least one magnetic field generator comprises at least one electromagnet and a source of electrical current in electrical communication with the electromagnet for delivering current to the electromagnet, the at least one electromagnet being operable upon the delivery of electrical current thereto to generate a magnetic field.

30. Apparatus as set forth in claim 28 further comprising a web support positioned for contiguous relationship with the material web upon movement of the web in said predetermined direction, the at least one magnetic field generator being operable to generate a magnetic field which urges the material web toward the web support upon movement of the web in said predetermined direction.

31. Apparatus as set forth in claim 28 wherein the web support comprises a roll rotatable relative to the predetermined direction of movement of the material web, the at least one magnetic field generator comprising at least one electromagnet and a source of electrical current in electrical communication with the at least one electromagnet for delivering electrical current thereto.

32. Apparatus as set forth in claim 31 wherein the at least one electromagnet is secured to the roll for concurrent movement with the roll upon rotation thereof.

33. Apparatus as set forth in claim 32 wherein the roll has an outer surface positioned for contiguous relationship with the web upon movement of the web in the predetermined direction, the at least one electromagnet defining at least part of the outer surface of the roll.

34. Apparatus as set forth in claim 30 wherein the apparatus is capable of moving the web in contiguous relationship with the web support generally from a first position of the web support to a second position thereof, the magnetic field generator being operable to generate a first magnetic field at said first position to urge the web toward the web support and being further operable to generate a second magnetic field at said second position, the second magnetic field being substantially weaker than said first magnetic field to facilitate release of the web from the web support.

35. Apparatus as set forth in claim 30 wherein the apparatus is capable of moving the web in contiguous relationship with the web support generally from a first position of the web support to a second position thereof, the magnetic field generator being operable to generate a magnetic field at said first position to urge the web toward the web support, said magnetic field being one of dissipated and reversed at said second position to facilitate release of the web from the web support.

36. Apparatus as set forth in claim 30 wherein the material web has a longitudinal axis, a lateral axis and laterally opposite side edges, said at least one magnetic field generator being operable to urge the web laterally outward relative to the longitudinal axis thereof.

37. Apparatus as set forth in claim 36 wherein the magnetic field is a first magnetic field, said at least one magnetic field generator being operable to generate a second magnetic field in opposed relationship with the first magnetic field whereby the first and second magnetic fields urge the side edges of the material web in laterally opposite directions to thereby configure the material web in a generally taut configuration.

38. Apparatus as set forth in claim 37 further comprising a web support positioned for contiguous relationship with the material web upon movement of the web in said predetermined direction, the first and second magnetic fields being generated upstream of the web support in said predetermined direction to configure said web in a generally taut configuration before the web is moved into contiguous relationship with the web support.

39. Apparatus as set forth in claim 28 wherein the at least one magnetic field generator comprises at least one permanent magnet.
